(19) United States
(12) Patent Application Publication
(10) Pub. No.: US 2023/0414919 A1
(43) Pub. Date: Dec. 28, 2023

(54) CANNULA SYSTEM

(71) Applicant: ABIOMED, Inc., Danvers, MA (US)

(72) Inventors: Alexander Ship, Danvers, MA (US); Emmanuel Ilongo, Danvers, MA (US); Vincent Roy, Danvers, MA (US); Sonya Bhavsar, Danvers, MA (US)

(73) Assignee: ABIOMED, Inc., Danvers, MA (US)

(21) Appl. No.: 17/852,201

(22) Filed: Jun. 28, 2022

Related U.S. Application Data
(60) Provisional application No. 63/355,355, filed on Jun. 24, 2022.

(51) Int. Cl. A61M 60/13; A61M 60/216; A61M 60/416; A61M 60/174; A61M 60/859; A61M 60/508; A61M 60/816

(52) U.S. Cl. CPC A61M 60/13; A61M 60/216; A61M 60/416; A61M 60/174; A61M 60/859; A61M 60/508; A61M 60/816; A61M 25/0069

(57) ABSTRACT

A system and method for percutaneous ventricular assist devices are provided, where the percutaneous ventricular assist devices include a pump assembly and a cannula assembly. The pump assembly includes one or more connectors at or near a distal end. The cannula assembly includes a cannula and an inflow cage, as well as a mating connector engageable with the connectors of the pump assembly such that the first cannula assembly is attachable to the pump assembly. In various embodiments, the cannula assembly may be fixedly attached or removably attached from the pump assembly.

### CROSS-REFERENCE TO RELATED APPLICATION

The present application claims priority to U.S. Provisional Pat. App. No. 63/355,355, filed Jun. 24, 2022, the entirety of which is incorporated by reference herein.

### TECHNICAL FIELD

This disclosure relates to medical devices, and more specifically to percutaneous ventricular assist devices with cannula assemblies for transferring a fluid into, out of, or through a body cavity, such as a blood vessel or the heart.

### BACKGROUND

Blood pumps of different types are known, such as axial blood pumps, centrifugal blood pumps, or mixed-type blood pumps, where the blood flow is caused by both axial and radial forces. One such example of a blood pump is the Impella® line of blood pumps (e.g., Impella 2.5®, Impella CP®, Impella 5.5®, etc.), which are products of Abiomed of Danvers. Intravascular blood pumps may be inserted into a patient's vessel, such as via the aorta, by means of a catheter.

### BRIEF SUMMARY

In some embodiments, a mechanical circulatory support device (MCS) such as a percutaneous ventricular assist device (PVAD) is provided. The PVAD (which may be, e.g., a percutaneous catheter-based heart pump) may include a pump assembly and a first cannula assembly. The pump assembly may have one or more connectors at or near a distal end. The first cannula assembly may include a first cannula and a first inflow cage, where a proximal end of the first cannula assembly may include one or more mating connectors engageable with the one or more connectors of the pump assembly such that the first cannula assembly is attachable to the pump assembly.

In some embodiments, the first cannula assembly may be configured to be attached to the pump assembly by a clinician prior to a medical procedure. In some embodiments, the first cannula assembly may be removably attachable to the pump assembly.

In some embodiments, the PVAD may include a pigtail operably connected to a distal portion of the first cannula assembly. In some embodiments, the pigtail may be fixedly attached to the distal end of the first cannula assembly such that the first cannula assembly and pigtail are attachable to the pump assembly as a single coupled assembly. In some embodiments, the pigtail may be fixedly attached to the first inflow cage. In some embodiments, the pigtail may be removably attachable to the first inflow cage.

In some embodiments, the PVAD may include a second cannula assembly that may include a second cannula and a second inflow cage. The second cannula assembly may be different from the first cannula assembly. The second cannula assembly may have a distal end and a proximal end, where the proximal end of the second cannula assembly may include one or more mating connectors engageable with the connectors of the pump assembly such that the second cannula assembly is attachable to the pump assembly. In some embodiments, one of the first cannula assembly and the second cannula assembly may be configured to be attached to the pump assembly by a clinician prior to a medical procedure.

In some embodiments, the pump assembly may include a motor operably connected to an impeller, the impeller configured to cause blood to flow through the first inlet cage, the cannula assembly, or a combination thereof.

In some embodiments, each mating connector may include a threaded portion. In some embodiments, each mating connector, each connector, or both may have a coating comprising an additive (such as silicone).

In some embodiments, one mating connector may include a detent, and one connector may include at least one wall defining an opening extending at least partially though the wall, the opening being configured to receive at least a portion of the detent. In some embodiments, each mating connector may include a detent, and each connector may include at least one wall defining an opening extending at least partially though the wall, each opening configured to receive at least a portion of the detent of one of the mating connectors. In some embodiments, each mating connector further comprises a silicone gasket, the detent being positioned between the silicone gasket and the connector.

In some embodiments, each mating connector, each connector, or both may include a magnetic element.

In some embodiments, a system may be provided. The system may include a PVAD as disclosed herein, and a controller configured to control the PVAD.

In some embodiments, a kit may be provided. The kit may include a pump assembly, a first cannula, and a second cannula that is different from the first cannula. The pump assembly may have one or more connectors at or near a distal portion. The first cannula assembly may have one or more mating connectors at or near a proximal portion of the first cannula assembly. The one or more mating connectors of first cannula assembly may engage with the one or more connector of the pump assembly such that the first cannula assembly is coupled to the pump assembly. The second cannula assembly may have one or more mating connectors at or near a proximal portion of the second cannula assembly.

In some embodiments, the first cannula assembly may be configured to be attached to the pump assembly by a clinician prior to a medical procedure. In some embodiments, the first cannula assembly may be removably attachable to the pump assembly.

The kit may include a pigtail assembly. The pigtail assembly may be configured to be connected to a distal portion of the first cannula assembly, the second cannula assembly, or both. In some embodiments, the pigtail may be fixedly attached to the distal end of the first cannula assembly such that the first cannula assembly and pigtail are attachable to the pump assembly as a single coupled assembly. In some embodiments, the pigtail is fixedly attached to an inflow cage on the first and/or second cannula assembly. In some embodiments, the pigtail is removably attachable to a first inflow cage on the first and/or second cannula assembly.

In some embodiments, the first cannula assembly may include a first cannula and a first inflow cage, and the second cannula assembly may include a second cannula and a second inflow cage. In some embodiments, the first cannula is longer than the second cannula. In some embodiments, the first cannula may be curved. In some embodiments, the second cannula may be straight.

The kit may include a third cannula assembly including a third cannula and a third inflow cage. The third cannula assembly may have one or more mating connectors, wherein the third cannula assembly is different from the first and/or the second cannula assemblies.

In some embodiments, each mating connector may include a threaded portion. In some embodiments, each mating connector, each connector, or both may have a coating comprising an additive (such as silicone).

In some embodiments, one mating connector may include a detent, and one connector may include at least one wall defining an opening extending at least partially though the wall, the opening being configured to receive at least a portion of the detent. In some embodiments, each mating connector may include a detent, and each connector may include at least one wall defining an opening extending at least partially though the wall, each opening configured to receive at least a portion of the detent of one of the mating connectors. In some embodiments, each mating connector further comprises a silicone gasket, the detent being positioned between the silicone gasket and the connector.

In some embodiments, each mating connector, each connector, or both may include a magnetic element.

The kit may include a controller configured to control the pump assembly.

In some embodiments, the kit may include an inflow assembly. The inflow assembly may be attachable to the first cannula assembly and/or the second cannula assembly.

In some embodiments, the first cannula assembly may include a sensor.

In some embodiments, a method for providing a PVAD, having a pump assembly and a plurality of cannula assemblies is provided. The method may include selecting a first cannula assembly of the plurality of cannula assemblies based on a characteristic of a patient. The method may also include attaching the first cannula assembly to the pump assembly by connecting one or more connectors of the first cannula assembly to one or more connectors of the pump assembly. In some embodiments, the method may include inserting at least a portion of the PVAD into a patient.

In some embodiments, the plurality of cannula assemblies may include an inflow assembly with a connector coupled to a proximal portion of the inflow assembly configured to connect to a mating connector coupled to a distal portion of a different cannula assembly.

In some embodiments, the PVAD may include a pigtail assembly configured to be connected to a distal portion of the first cannula assembly.

In some embodiments, the pigtail may be fixedly attached to the distal end of the first cannula assembly such that the first cannula assembly and pigtail are attachable to the pump assembly as a single coupled assembly. In some embodiments, the pigtail is fixedly attached to an inflow cage on the first and/or second cannula assembly. In some embodiments, the pigtail is removably attachable to a first inflow cage on the first and/or second cannula assembly.

In some embodiments, the first cannula assembly may include a first cannula and a first inflow cage, and the second cannula assembly may include a second cannula and a second inflow cage. In some embodiments, the first cannula is longer than the second cannula. In some embodiments, the first cannula may be curved. In some embodiments, the second cannula may be straight.

In some embodiments, the pump assembly may include a motor operably connected to an impeller, the impeller configured to cause blood to flow through the first inlet cage, at least one of the cannula assemblies, or a combination thereof.

In some embodiments, the PVAD may include a controller configured to control the pump assembly. In some embodiments, the first cannula assembly may be attached to the pump assembly by a clinician prior to a medical procedure. In some embodiments, the first cannula assembly is removably attachable to the pump assembly.

In some embodiments, a percutaneous ventricular assist device may be provided. The PVAD may include a pump assembly having a distal end and proximal end. The PVAD may include a first cannula assembly having a first cannula and a sensor. The first cannula assembly may have a distal end and a proximal end. The proximal end may be attached to the pump assembly. The distal end may have one or more connectors. The PVAD may include a second cannula assembly, which may have a second cannula and a first inflow cage. The second cannula may include one or more mating connectors engageable with the one or more connectors of the first cannula assembly such that the second cannula assembly may be attachable to the first cannula assembly by a clinician before a medical procedure.

In some embodiments, the PVAD may include a pigtail, which may be operably coupled to a distal portion of the second cannula assembly. In some embodiments, the PVAD is a catheter-based heart pump. In some embodiments, the pump assembly may include a motor operably connected to an impeller. The impeller may be configured to cause blood to flow through a first inlet cage of the second cannula assembly, the first cannula assembly, the second cannula assembly, or a combination thereof. In some embodiments, the one or more connectors may include a threaded connector.

In some embodiments, the one or more mating connectors, the one or more connectors, or both may have a coating. The coating may include an additive. The additive may include silicone.

In some embodiments, the one or more mating connectors may include a detent. In some embodiments, one or more connectors may include at least one wall defining an opening extending at least partially though the wall. The opening may be configured to receive a portion of the detent.

In some embodiments, each mating connector, each connector, or both may include a magnetic element.

In some embodiments, a method may be provided. The method may include providing a ventricular assist device having a pump assembly and a first cannula assembly, where the first cannula assembly may have a first cannula. The step of providing may include reducing a length of the first cannula of the first cannula assembly to achieve a prescribed length of the first cannula. In some embodiments, reducing a length of the first cannula may include removing a length of the first cannula. In some embodiments, the step of providing may include attaching the first cannula assembly to the pump assembly via one or more connectors and one or more corresponding mating connectors. In some embodiments, the first cannula assembly includes an inlet cage. In some embodiments, the step of providing may include attaching a second cannula assembly to the first cannula assembly via one or more connectors and one or more corresponding mating connectors. In some embodiments, the second cannula assembly may include an inlet cage. In some embodiments, each of the first and/or second cannula assembly may include a sensor. In some embodiments, the first cannula may be permanently attached to the pump assembly.

### BRIEF DESCRIPTION OF DRAWINGS

FIG. 1 is a schematic of an embodiment of a percutaneous ventricular assist device.

FIG. 2A is a partial cross-sectional schematic of an embodiment of a threaded connector and mating connector.

FIG. 2B is a cross-sectional schematic of an embodiment of a connector and mating connector utilizing a detent.

FIG. 2C is a partial cross-sectional schematic of an embodiment of a connector and mating connector utilizing a magnet.

FIG. 3A is a schematic of an embodiment of a percutaneous ventricular assist device.

FIG. 3B is a schematic of an embodiment of a percutaneous ventricular assist device.

FIG. 3C is a schematic of an alternative embodiment of a percutaneous ventricular assist.

FIG. 3D is a schematic of yet another embodiment of a percutaneous ventricular assist device.

FIG. 4 is a depiction of a blood pump laid through the aorta, extending through the aortic valve into the left ventricle.

FIG. 5 is a partial cross-sectional schematic of an embodiment of a third cannula assembly.

FIG. 6 is a flowchart of an embodiment of a method.

FIG. 7 is a schematic of an embodiment of a percutaneous ventricular assist device.

### DETAILED DESCRIPTION

Blood pumps of different types are known, such as axial blood pumps, centrifugal blood pumps, or mixed-type blood pumps, where the blood flow is caused by both axial and radial forces. One such example of a blood pump is the Impella® line of blood pumps (e.g., Impella 2.5®, Impella CP®, Impella 5.5®, etc.), which are products of Abiomed of Danvers. Intravascular blood pumps may be inserted into a patient's vessel, such as via the aorta, by means of a catheter.

Current catheter-based pumps utilize a cannula that is designed to generally fit most patients for a particular indication. Accordingly, such devices, while designed to cover a broad range of implementations, cannot be modified to satisfy a wide range of patients suffering from the varying conditions. As such, this may eliminate the ability for a clinician to provide more personalized care and/or allow treatment of more niche clinical indications (e.g., pediatric, direct right ventricle support, and non-cardiac support, such as renal).

In view of the above, the inventors have recognized the benefit of a system that is capable of being configured by a treating clinician to satisfy more patients and/or procedures.

Disclosed herein are mechanical circulatory support (MCS) devices (e.g., percutaneous ventricular assist devices or PVADs) having specific combinations of pump assemblies, connectors, cannula assemblies, and/or inflow assemblies in order to provide more flexibility for medical staff in treating a particular condition in a particular individual. In that regard, in some embodiments, the devices may include modular units which may be configurable by the clinician (e.g., a surgeon and/or interventional cardiologist) to appropriately fit the patient before being implanted.

In some embodiments, the PVAD may include a catheter-based blood pump or heart pump providing mechanical circulatory support.

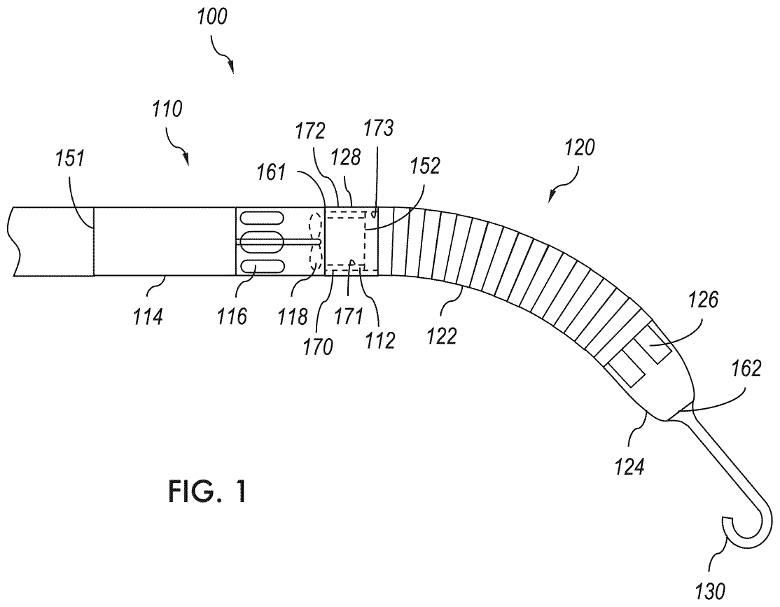

Turning now to the figures, and as seen in FIG. 1, in some embodiments, a percutaneous ventricular assist device may include a blood pump 100 having a pump assembly 110 and a cannula assembly 120.

In some embodiments, the cannula assembly includes a proximal end 161 and a distal end 162. In some embodiments, the cannula assembly may have a cannula 122 and an inlet cage 124 (also referred to herein as an inflow cage). In some embodiments, the inlet cage 124 may be positioned distal to the cannula 122.

In some embodiments, the cannula 122 may have an inner surface 173 and an outer surface 172, with a sidewall therebetween. The inner surface may define a lumen extending from a proximal end of the cannula to a distal end of the cannula. As will be appreciated, in such embodiments, fluid may be transferred through the lumen during use of the device.

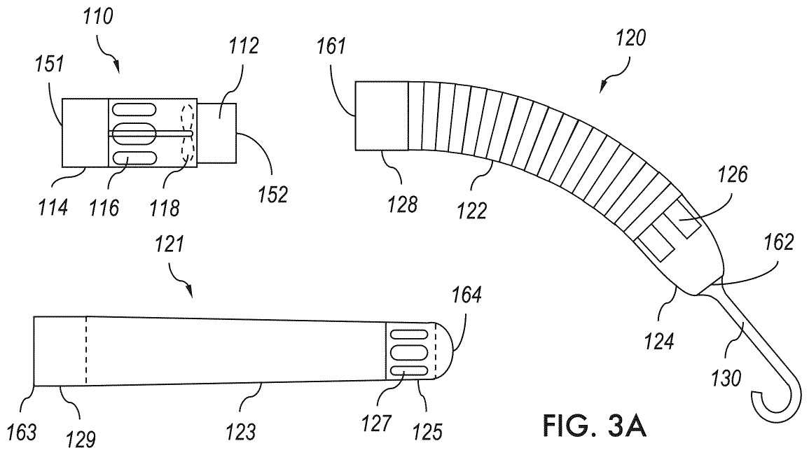

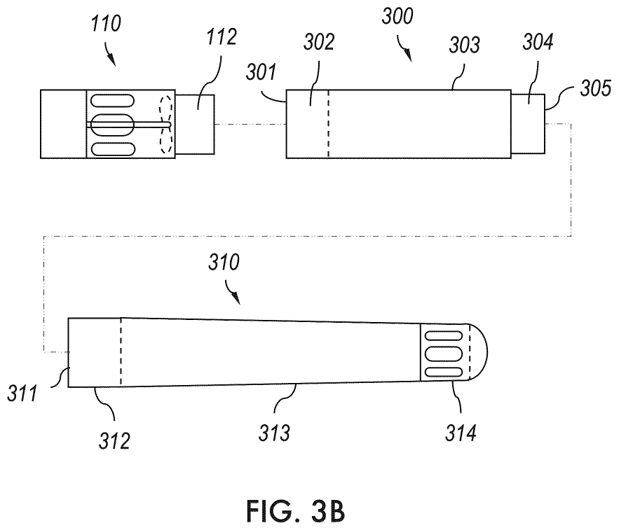

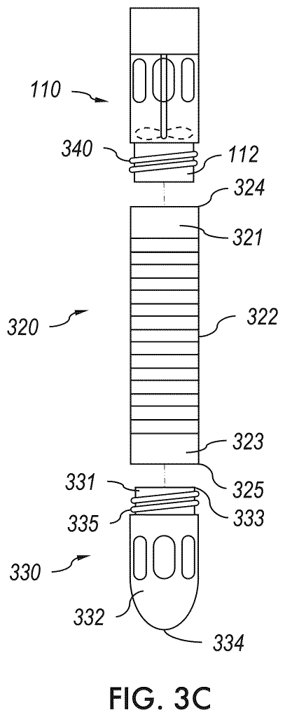

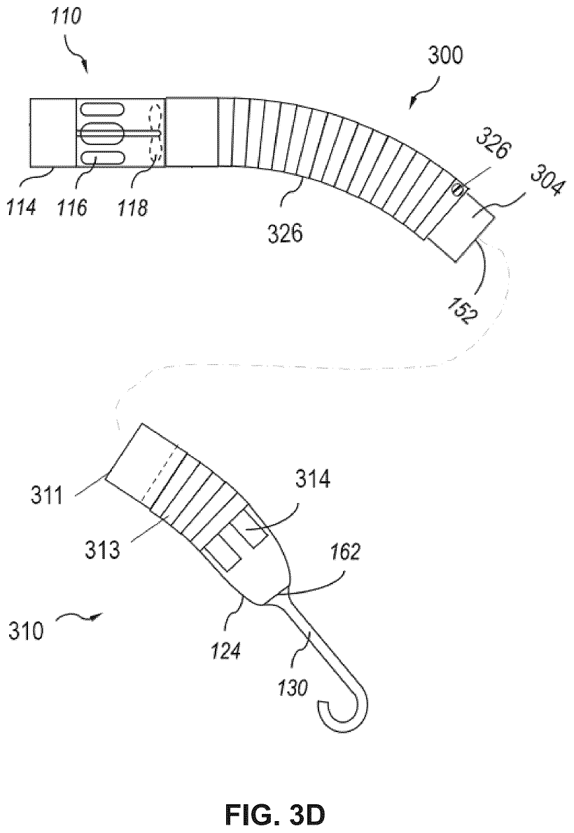

The shape of the cannula 122 may vary as desired. In some embodiments, the cannula may be curved (see, e.g., FIG. 1). In some embodiments, the cannula may be straight (see, e.g., cannula 123 in FIG. 3A). As will be appreciated, in some embodiments, the cannula also may have a portion that is straight and another portion that is curved. The cannulas also may have different diameters.

As also shown in FIG. 1, the pump assembly may have a proximal end 151 and a distal end 152. In some embodiments, the pump assembly may include motor 114 operably connected to an impeller 118. In some embodiments, the impeller may be configured to cause blood to flow through openings 126 in the inlet cage 124, through the cannula 122, and out through openings 116 in the pump assembly 110.

As will be appreciated, in some embodiments, the pump assembly may be configured such that the blood flow may be reversed—that is, in some embodiments, the impeller may be configured to cause blood to flow through openings 116 in the pump assembly 110, through the cannula 122, and out through openings 126 in the inlet cage 124.

The pump assembly may have an inner surface 171 and an outer surface 170 with a sidewall therebetween. The inner surface may define a lumen that extends from the distal end of the pump assembly at least partially towards the proximal end.

As described herein, the pump may include a modular unit such that a clinician may appropriately configure the pump for a particular patient and/or indication. In some embodiments, the pump may be configured such that the clinician may attach a desired cannula assembly to the pump assembly to prepare the pump. In that regard, the pump assembly may have a connector that engages with a corresponding connector on the cannula. For example, the pump assembly may have a connector 112 at the distal end of the pump assembly configured to engage with a mating connector 128 at the proximal end of the cannula assembly. The connector and the mating connector may be coupled together in some embodiments to form the pump assembly. In some embodiments, the connector and mating connector may be coupled such that the cannula assembly and pump assembly may be fixedly attached together.

In some embodiments, an inner surface 172 of the cannula assembly may interact with an outer surface 170 of the pump assembly. In some embodiments, an outer surface 173 of the cannula assembly may interact with an inner surface 171 of the pump assembly.

The pump assembly 110 may have one or more connectors at or near the distal end, and the cannula assembly 120 may have one or more mating connectors at or near the proximal end that are engageable with the one or more connectors of the pump assembly such that the first cannula assembly is attachable to the pump assembly. In some embodiments, the mating connector is configured to be fixedly attached to the connector. In some embodiments, the mating connector is configured to be removably attached to the connector.

In some embodiments, the first cannula assembly may be configured to be attached to the pump assembly by a clinician prior to a medical procedure.

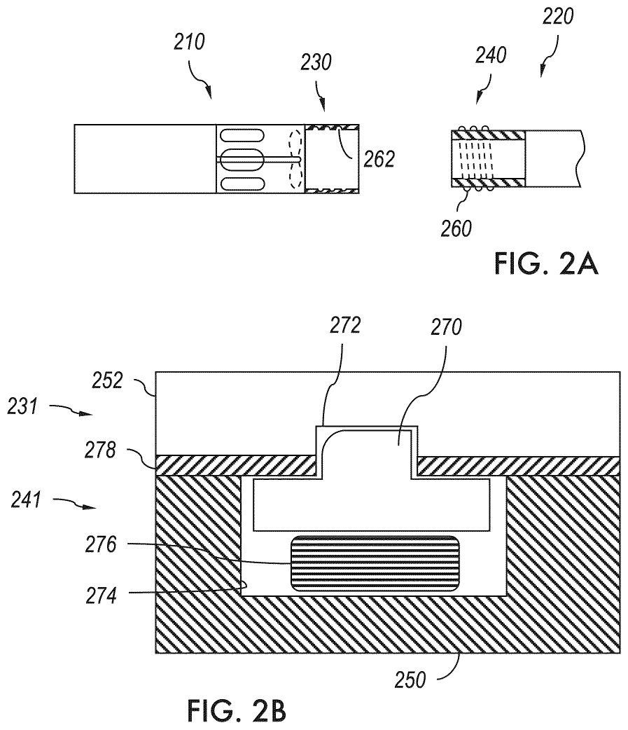

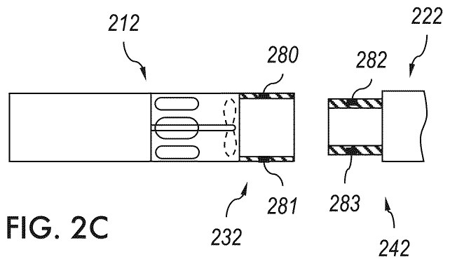

Referring to FIG. 2A, in some embodiments, the cannula assembly 220 may have a mating connector 240 that includes one or more threads 260 (each thread may include a separate mating connector in some embodiments) The pump assembly 210 may have a connector 230 that includes one or more recesses 262 (each recess being a separate connector in some embodiments) configured to receive the threads. It will be readily understood that the reverse is also envisioned, where the pump assembly may have a connector that includes one or more threads, and the cannula assembly may have a mating connector that includes the recesses for receiving the threads. Further, it will be readily understood that, while FIG. 2A shows the connector 230 would encompass the mating connector portion when assembled, the reverse is easily achievable.

As will be appreciated in view of FIG. 2, in some embodiments, to connect the pump assembly and the cannula assembly, the cannula may be threaded onto the pump assembly. In such embodiments, the shape and size of the one or more threads may correspond to the shape and size of the one or more recesses.

In some embodiments, each mating connector, each connector, or both have a coating comprising an additive. In some embodiments, the coating comprises silicone.

Referring to FIG. 2B, in some embodiments, a mating connector 241 may include a detent 270. In such configurations, the connector 231 may include a connector wall 252 defining a first opening 272 extending at least partially through the connector wall, where the first opening 272 may be configured to receive at least a portion of the detent 270. As will be appreciated, in some embodiments, the first opening may be configured to receive the entire detent. In some embodiments, the mating connector may include more than one detent (each detent being a separate mating connector in some embodiments), and the connector may include more than one opening (with each opening in the wall being a separate connector in some embodiments). As will be appreciated, the shape and size of the detent may correspond to the shape and size of the detent. As will be further appreciated, although the connector is show as being one or more openings to receive a mating connector having one or more detents, in some embodiments, the mating connector may include one or more openings that receive one or more detents of the connector.

In some embodiments, the connector may include a mating connector wall 250 defining a second opening 274 extending at least partially through the mating connector wall, where the opening 274 is hold at least a portion of the detent 270. In some embodiments, the opening 274 may be partially covered by a collar 278, the collar configured to allow at least a portion of the detent to extend out of the mating connector portion 241 and into the first opening of the connector portion 231 when the mating connector and connector are properly positioned.

In some embodiments, the mating connector may include a gasket 276, such as a silicone gasket. The gasket may be arranged such that the detent 270 is positioned between the gasket 276 and the connector 231.

In various embodiments, one or more detents may be utilized. In some embodiments, one mating connector comprises a detent, and one connector comprises at least one wall defining an opening extending at least partially though the wall, the opening configured to receive a portion of the detent. In some embodiments, each mating connector comprises a detent, and each connector comprises at least one wall defining an opening extending at least partially though the wall, the opening configured to receive a portion of the detent.

In other embodiments, the pump assembly and the cannula assembly may be held together magnetically. For example, in some embodiments, the connector may have one or more magnets and/or one or more ferromagnetic portions that a magnet may couple to. In some embodiments, the mating connector may have one or more magnets and/or one or more ferromagnetic portions that a magnet may couple to. In some embodiments, the one or more magnets may include permanent magnets. In some embodiments, one or more magnets may include electromagnets.

Referring to FIG. 2C, a pump assembly 212 may have a connector portion 232 with a magnet 280, 281. The cannula assembly 222 may have a mating connector portion 242 that includes one magnet 282 and a ferromagnetic portion 283. In this manner, the first magnet 280 in the connector will couple to the magnet 282 in the mating connector, and the second magnet 281 in the connector will couple to the ferromagnetic portion 283, when the mating connector is inserted into the connector. If more than one magnet or ferromagnetic portion is present, each magnet or ferromagnetic portion may include a separate connector or mating connector as appropriate.

As will be appreciated, the mating connector and connector may have other suitable engagements for attaching the cannula assembly to the pump assembly (e.g., press fit and or snap fit arrangements). As will be further appreciated, in some embodiments, attaching the cannula assembly and the pump assembly (or other suitable assemblies) to one another may lock the assemblies together such that they may not be disconnected during use of the pump.

In some embodiments, there may be a 1:1 correlation of connectors to mating connectors. For example, in some embodiments, there may be a single detent that is received in a single opening of a wall. In other embodiments, the number of connectors and mating connectors may vary. For example, the correlation may be between 3:1-1:3 in some embodiments. In still other embodiments, the correlation is 10:1-1:10.

As will be appreciated, in some embodiments, more than one type of connector may be used to connect the pump assembly and cannula assembly of the pump, such as for redundancy. For example, in some embodiments, the mating connector may include both threads and a detent that engage with a connector having corresponding threads and a wall opening.

Referring back to FIG. 1, in some embodiments, the pump may include a pigtail 130. The pigtail may be operably connected to a distal portion of the cannula assembly 120.

In some embodiments, the pigtail may be fixedly attached to the distal end 162 of the cannula assembly 120 such that the cannula assembly 120 and pigtail 130 are attachable to the pump assembly as a single coupled assembly. In some embodiments, the pigtail 130 may be fixedly attached to the inflow cage 124.

In some embodiments, the pigtail 130 may be removably attachable to the inflow cage 124. This may be accomplished in various manners. For example, a proximal end of the pigtail may include a snap connection, an interference fit, a screw type connector, or any other suitable reversible or detachable connector, and the cannula assembly would include the appropriate counterpart for the providing the connection.

As described herein, the pump may include a modular system, such that a clinician can choose the desired style and fit of the pump. Referring to FIG. 3A, in some embodiments, the PVAD may include a second cannula assembly 121 having a proximal end 163 and a distal end 164. The second cannula assembly may include a second cannula 123 and a second inflow cage 125. The second inflow cage may have one or more openings 127 configured to allow blood to enter or exit the second cannula 123. The second cannula assembly may include a mating connector 129 that is also engageable with the connector of the pump assembly such that the second cannula assembly is attachable to pump assembly. In that regard, each of the first and second cannulas may have the same mating connector for engaging with the connector on the pump assembly.

In some embodiments, at least one characteristic of the second cannula assembly 121 is different than the first cannula assembly 120. For example, in some embodiments, the second cannula assembly may have a straight cannula, while the first cannula assembly may have a curved cannula (or at least a partially curved cannula). In some embodiments, the maximum outer diameter of the second cannula assembly may be different from the maximum outer diameter of the first cannula assembly. In some embodiments, the distance from proximal end to distal end of the second cannula assembly (e.g., a length of the proximal cannula) is different from the distance from proximal end to distal end of the first cannula assembly.

In some embodiments, either the first cannula assembly or the second cannula assembly is configured to be attached to the pump assembly by a clinician prior to a medical procedure. In this regard, the physician may choose the appropriate length and/or style of cannula for the appropriate procedure and/or patient.

In some embodiments, a system for ventricular assistance is provided. The system may include an embodiment of a percutaneous ventricular assist device as disclosed herein, and a controller configured to control the percutaneous ventricular assist device.

In some embodiments, the pump device may include more than two assemblies that can be coupled together. For example, as shown in FIG. 3B, a connector portion 112 at a distal end of a pump assembly 110 may be coupled to a mating connection 302 at a proximal end 301 of a first cannula assembly 300 comprising a cannula 303. A connector 304 at a distal end 305 of the cannula 303 may then be coupled to a mating connector portion 312 at a proximal end 311 of a cannula 313 of a second, cannula assembly 310, an inflow cannula assembly. The inflow cannula assembly 310 may include an inflow cage 314 coupled to a distal end of a cannula 313. As will be understood, various designs of these cannula assemblies can be utilized. Further, while a single intermediate cannula assembly 300 (e.g., the first cannula assembly) is shown in FIG. 3B, multiple such cannulas could be coupled together before connecting to inflow assembly 310 (also called an inlet assembly).

In some embodiments, as shown in FIG. 3D, the first cannula assembly may include a sensor 326, e.g., a pressure or flow sensor, that is configured to measure one or more parameters of the patient and/or the pump. In some embodiments, the sensor may be used to help determine whether or not the pump is operating correctly and/or providing appropriate support for the patient. For example, the sensor may assist in determining the proper position of the pump in the patient and/or the appropriate speed of the pump. In such embodiments, the first cannula assembly may be configured such that a length of the first cannula would provide optimal position of the sensor in the body (e.g., in the heart). As will be appreciated, the pump also may have a second sensor located in another suitable position.

The combination of cannulas and inflow cage may be combined in different fashions than that shown in FIG. 3B. For example, as seen in FIG. 3C, an inflow assembly 330 may include an inflow cage 332 and a connector portion 331. In some embodiments, threads 335 are connectors coupled to a proximal portion of the inflow assembly. The threads also may be integrally formed with the inflow cage. In such embodiments, the connectors are configured to connect to a mating connector in a mating connector portion 323 (the connector coupled to a distal portion of a first cannula assembly—here, cannula assembly 320). In this embodiment, cannula assembly 320 includes a cannula 322 and a mating connector 321 at a proximal end 324 of the cannula assembly, the mating connector configured to connect to a connector coupled to a distal portion of the pump assembly, here threads 340, in connector portion 112.

In some embodiments, as shown in FIG. 3D, the first cannula assembly 300 may be attached (e.g., fixedly and/or permanently attached) to the pump assembly, with the only the cannula 300 assembly having a connector 304 that couples to a mating connector 311 on the second, inflow cannula assembly 310. As will be appreciated, in such embodiments, the inflow cannula assembly 310 may be formed having different lengths, with the clinician choosing the desired inflow cannula assembly to attach to the pump assembly 300.

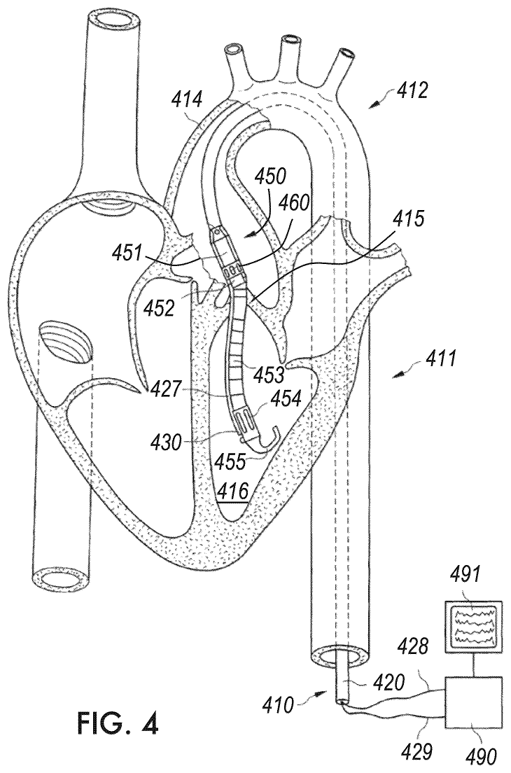

FIG. 4 illustrates an embodiment where an intravascular blood pump having a catheter 410 is introduced into a patient's heart. For example, as show in this figure, the blood pump may be inserted into the descending aorta 411 retrograde in some embodiments. As is known, the descending aorta is part of the aorta 412 which first ascends from the heart and then descends and has the aortic arch 414. At the beginning of the aorta 412 there is located the aortic valve 415 which connects the left ventricle 416 to the aorta 412 and through which the intravascular blood pump may extend. As will be appreciated, blood pumps may be inserted into other suitable portions of the body.

As will be appreciated in view of the above, the intravascular blood pump assembly may include a pump assembly 450 coupled at a distal end of the catheter hose 420 and having a motor section 451 and a pump assembly 452 disposed at an axial distance therefrom, as well as a cannula assembly 453 protruding in the distal direction from the distal end of the pump assembly 452. The cannula assembly may include an inflow cage 430 located at its end, the inflow cage having openings 454, as described above, or the pump assembly may include an inflow cage assembly. Distally of the inflow cage 454 there is provided a tip 455, which may be a flexible tip which can be configured for example as a “pigtail” or in a J shape. Through the catheter hose 420 there extend different lines and devices which may be important for operating the pumping device 450. As will be appreciated in view of the above, the clinician may select the appropriate pump assembly, first and/or second catheter assembly, and/or inflow assembly and assemble the desired pump for implanting in the patient as is shown.

In some embodiments, the pump may include one or more electrical connections 428 and/or one or more optical fibers 429 that are attached at their proximal end to a controller 490. The optical fiber(s) may be, e.g., part of an optical sensor (such as a pressure sensor) whose sensor heads may be located in the vicinity of the inflow cage 430 (see, e.g., sensor 326 in FIG. 3D), on or in the pump section, or a combination thereof. In some embodiments, the controller converts electrical or optical signals into information which can then be displayed, e.g., on a display screen 491.

In some embodiments, a PVAD kit may be provided. Referring to FIG. 3A, in some embodiments, the kit may include a pump assembly 110 having a connector portion 112 that may include one or more connectors at or near a distal portion. In some embodiments, the kit may include a first cannula assembly 120 having a mating connector portion 128 that may include one or more mating connectors at or near a proximal portion of the first cannula assembly. In some embodiments, the kit may include a second cannula assembly (e.g., second cannula assembly 121 in FIG. 3A, second cannula 320 in FIG. 3C, and/or second inflow cannula 310 in FIGS. 3B and 3D) having a corresponding mating connector at or near a proximal portion of the second cannula assembly. In still other embodiments, the kit may include an inflow cage assembly with one or more connectors or mating connectors.

In some embodiments, the second cannula assembly may be the same as the first cannula assembly. In some embodiments, the second cannula assembly may be different from the first cannula assembly.

In some embodiments, the one or more mating connectors of the first cannula assembly may be configured to engage with the one or more connector of the pump assembly such that the first cannula assembly is or can be coupled to the pump assembly.

In some embodiments, the one or more mating connectors of the second cannula assembly may be configured to engage with the one or more connector of the pump assembly such that the second cannula assembly is or can be coupled to the pump assembly.

In some embodiments, the kit may include a pigtail assembly 130 configured to be connected to a distal portion of the first cannula assembly, the second cannula assembly, or both.

In some embodiments, the pigtail may be fixedly attached to the distal end 162 of the cannula assembly 120 such that the cannula assembly 120 and pigtail 130 are attachable to the pump assembly as a single coupled assembly. In some embodiments, the pigtail 130 may be fixedly attached to the first inflow cage 124. In some embodiments, the pigtail 130 may be fixedly attached to the second inflow cage 125.

In some embodiments, the pigtail 130 may be removably attachable to the first inflow cage 124. In some embodiments, the pigtail 130 may be removably attachable to the second inflow cage 125.

In some embodiments, at least one characteristic of the second cannula assembly 121 is different than the first cannula assembly 120. For example, in some embodiments, the second cannula assembly may have a straight cannula, while the first cannula assembly may have a curved cannula. In some embodiments, the maximum outer diameter of the second cannula assembly is different from the maximum outer diameter of the first cannula assembly. In some embodiments, the distance from proximal end to distal end of the second cannula assembly is different from the distance from proximal end to distal end of the first cannula assembly. In some embodiments, the first cannula is longer than the second cannula. In some embodiments, one of the first and second cannulas may include a sensor.

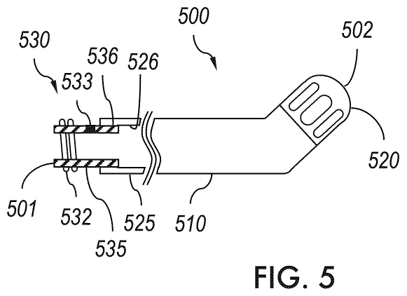

In some embodiments, the kit may include a third cannula assembly. As seen in FIG. 5, the third cannula assembly 500 may include a third cannula 510 and a third inflow cage 520. The third cannula assembly may include a mating connector portion 530. The mating connector portion may include one or more mating connectors 532, 533 (here, threads 532 and a magnet 530).

In some embodiments, at least one characteristic of the third cannula assembly 500 is different than the first cannula assembly 120, the second cannula assembly 121, or both. For example, in some embodiments, the second cannula assembly and third cannula assembly may each have a straight cannula, while the first cannula assembly may have a curved cannula. In some embodiments, the maximum outer diameter of the third cannula assembly is different from the maximum outer diameter of the first cannula assembly, the second cannula assembly, or both. In some embodiments, the distance from proximal end to distal end of the third cannula assembly is different from the distance from proximal end to distal end of the first cannula assembly, the second cannula assembly, or both. In some embodiments, the third cannula is longer than the first cannula, the second cannula, or both.

As seen in FIGS. 1, 2A, 2C, and 5, it will be understood that the mating connector portion of any disclosed cannula assembly may be related to its associated cannula in different ways. For example, referring to FIG. 5, in some embodiments, the sidewall 535 forming the mating connector 530 may be coupled to a surface of the cannula. For example, an external surface 536 of sidewall 535 being coupled (via an adhesive, thermal bonding, etc.) to an internal surface 526 of a sidewall 525 of the cannula 520. In other embodiments, a proximal portion of the sidewall forming the cannula may be considered the mating connector portion.

In some embodiments, each mating connector may include a threaded portion. In some embodiments, each mating connector, each connector, or both may have a coating comprising an additive (such as silicone).

In some embodiments, one mating connector may include a detent, and one connector may include at least one wall defining an opening extending at least partially though the wall, the opening being configured to receive at least a portion of the detent. In some embodiments, each mating connector may include a detent, and each connector may include at least one wall defining an opening extending at least partially though the wall, each opening configured to receive at least a portion of the detent of one of the mating connectors. In some embodiments, each mating connector further comprises a silicone gasket, the detent being positioned between the silicone gasket and the connector.

In some embodiments, each mating connector, each connector, or both may include a magnetic element.

In some embodiments, the kit may include a controller configured to control the pump assembly. In some embodiments, the first cannula assembly may be attached to the pump assembly by a clinician prior to a medical procedure. In some embodiments, the first cannula assembly is removably attachable to the pump assembly.

In some embodiments, a method for providing a percutaneous ventricular assist device having a pump assembly and a plurality of cannula assemblies can be utilized.

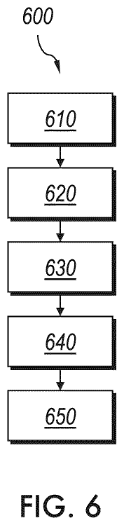

Referring to FIG. 6, the method 600 may include selecting 610 a first cannula assembly of the plurality of cannula assemblies based on a characteristic of a patient and/or a procedure being performed on the patient.

In some embodiments, the characteristic may include a diagnosis or testing result, an age of the patient, a size of the patient, and/or another condition of the patient.

The method may include attaching 620 the first cannula assembly to the pump assembly by connecting one or more connectors of the first cannula assembly to one or more connectors of the pump assembly.

In some embodiments, the method includes attaching a second cannula assembly (e.g., a cannula inflow assembly) to the first cannula assembly. In some embodiments, the method includes attaching an inflow assembly to the cannula assembly.

In some embodiments, the method may include inserting 630 some or all of the percutaneous ventricular assist device into a patient (see, e.g., FIG. 4).

In some embodiments, the plurality of cannula assemblies may include a second cannula assembly and/or a third cannula assembly. In some embodiments, the second cannula assembly includes a cannula and an inflow cage.

In some embodiments, the plurality of cannula assemblies comprises an inflow assembly with a connector coupled to a proximal portion of the inflow assembly, the connector configured to connect to a mating connector coupled to a distal portion of a different cannula assembly.

In some embodiments, the first cannula assembly and/or one of the second or third cannula may include a pigtail assembly configured to be connected to a distal portion of the first cannula assembly, the second cannula assembly, or both.

In some embodiments, the pigtail may be fixedly attached to the distal end of the cannula assembly such that the cannula assembly and pigtail are attachable to the pump assembly as a single coupled assembly. In some embodiments, the pigtail may be fixedly attached to the first inflow cage. In some embodiments, the pigtail may be fixedly attached to the second inflow cage.

In some embodiments, the pigtail may be removably attachable to the first inflow cage.

In some embodiments, at least one characteristic of the second cannula assembly is different than the first cannula assembly. For example, in some embodiments, the second cannula assembly may have a straight cannula, while the first cannula assembly may have a curved cannula. In some embodiments, the maximum outer diameter of the second cannula assembly is different from the maximum outer diameter of the first cannula assembly. In some embodiments, the distance from proximal end to distal end of the second cannula assembly is different from the distance from proximal end to distal end of the first cannula assembly.

In some embodiments, either the first cannula assembly or the second cannula assembly is configured to be attached to the pump assembly by a clinician prior to a medical procedure.

In some embodiments, the pump assembly may include a motor operably connected to an impeller. In some embodiments, the impeller may be configured to cause blood to flow through openings in an inlet cage, through a cannula, and out through openings in the pump assembly.

In some embodiments, the method 600 includes controlling 640 the pump assembly using a controller to cause blood to flow through one or more of the plurality of cannula assemblies of the assembled pump. In some embodiments, the method may include sensing one or more characteristics of the patient and/or pump via a sensor and controlling the pump based on the sensed data. For example, in some embodiments, controlling the pump may include changing a speed of the pump and/or changing a location of the pump.

In some embodiments, the method 600 includes removing 650 the pump assembly from the patient, such as after treatment has concluded.

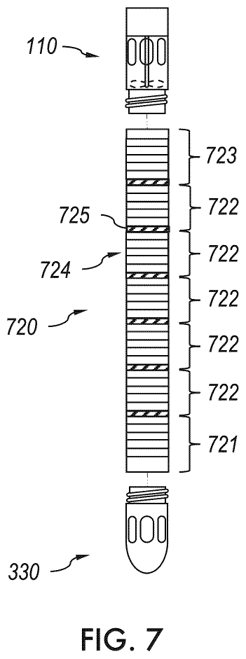

Although embodiments are shown and described in which the desired pump is formed via connecting one or more assemblies together (e.g., a pump assembly, a cannula assembly, and/or an inflow assembly), in other embodiments, the desired pump may be provided in other suitable manners. For example, referring to FIG. 7, in some embodiments, the pump may include a large cannula assembly 720 that may be reduced in size to accommodate that of the patient and/or procedure. For example, in some embodiments, the cannula may include on or more regions 721, 722, 723 that may be selectively removable to achieve the desired length. In some embodiments, a distal region 721 and/or one or more intermediate regions 722 may be removed. In some embodiments, a proximal region 723 and/or one or more intermediate regions 722 may be removed. In some embodiments, each region may include a first portion 725 that is intended to be cut through or otherwise used as a separation point, and a second portion 724 that is not intended to be cut through or otherwise used as a separation point. For example, in some embodiments, a clinician may cut and remove a length of the cannula assembly until the desired length is achieved. In other embodiments, the entire length of the cannula may be configured such that a clinician may cut and remove any desired length. In some embodiments, the cannula assembly may be fixedly attached to the pump assembly (e.g., not needing attachment via one or more connectors before use) such that the clinician need only remove the extraneous length of the cannula assembly before inserting the cannula assembly into the patient. As will be appreciated, the length of the cannula assembly also may be removed via other suitable manners, such as via snapping and/or peeling off a certain length of the cannula, by having one or more telescoping portions that are retractable into another portion of the cannula assembly and/or the pump assembly, or via other suitable manners.

As will be further appreciated, in some embodiments, the length of the cannula assembly may be adjusted, with the cannula assembly thereafter being attachable to the pump assembly and/or to an inflow assembly. For example, in some embodiments, the cannula assembly may be configured such that one or more connectors (e.g., mating connectors) are attachable to the cannula assembly such that the cannula assembly can thereafter be attached to the pump assembly. In this regard, the shortening of the cannula assembly may occur at the proximal end of the cannula assembly. In other embodiments, the distal end of the cannula assembly may be shortened, with the distal end of the cannula assembly having multiple connectors that can engage with an inlet assembly to attach the inlet assembly to the cannula assembly.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

What is claimed is:

1. A percutaneous ventricular assist device, comprising:
a pump assembly having a distal end and proximal end, the pump assembly having one or more connectors at or near the distal end; and
a first cannula assembly including a first cannula, the first cannula assembly having a distal end and a proximal end, the proximal end including one or more mating connectors engageable with the one or more connectors of the pump assembly; wherein the first cannula assembly is configured to be attached to the pump assembly by a clinician before a medical procedure.

2. The percutaneous ventricular assist device according to claim 1, wherein the first cannula assembly includes a first inflow cage attached to the first cannula assembly.

3. The percutaneous ventricular assist device according to claim 1, wherein the first cannula assembly is removably attachable to the pump assembly.

4. The percutaneous ventricular assist device according to claim 1, further comprising a pigtail operably connected to a distal portion of the first cannula assembly.

5. The percutaneous ventricular assist device according to claim 4, wherein the pigtail is fixedly attached to the distal end of the first cannula assembly such that the first cannula assembly and pigtail are attachable to the pump assembly as a single coupled assembly.

6. The percutaneous ventricular assist device according to claim 5, wherein the pigtail is fixedly attached to a first inflow cage of the first cannula assembly.

7. The percutaneous ventricular assist device according to claim 4, wherein the pigtail is removably attachable to a first inflow cage of the first cannula assembly.

8. The percutaneous ventricular assist device according to claim 1, further comprising a second cannula assembly including a second cannula and a second inflow cage;
wherein the second cannula assembly has a distal end and a proximal end, the proximal end of the second cannula assembly including one or more mating connectors is engageable with the connectors of the pump assembly such that the second cannula assembly is attachable to pump assembly;
wherein the second cannula assembly is different than the first cannula assembly; and
wherein the first cannula assembly or the second cannula assembly is configured to be attached to the pump assembly by a clinician prior to a medical procedure.

9. The percutaneous ventricular assist device according to claim 1, wherein the percutaneous ventricular assist device is a catheter-based heart pump.

10. The percutaneous ventricular assist device according to claim 8, wherein the pump assembly includes a motor operably connected to an impeller, the impeller configured to cause blood to flow through a first inlet cage of the first cannula assembly, the first cannula assembly, or a combination thereof.

11. The percutaneous ventricular assist device according to claim 1, wherein each mating connector comprises a threaded portion.

12. The percutaneous ventricular assist device according to claim 11, wherein each mating connector, each connector, or both have a coating comprising an additive.

13. The percutaneous ventricular assist device according to claim 12, wherein the additive comprises silicone.

14. The percutaneous ventricular assist device according to claim 1, wherein one mating connector comprises a detent, and one connector comprises at least one wall defining an opening extending at least partially though the wall, the opening configured to receive a portion of the detent.

15. The percutaneous ventricular assist device according to claim 1, wherein each mating connector comprises a detent, and each connector comprises at least one wall defining an opening extending at least partially though the wall, the opening configured to receive a portion of the detent.

16. The percutaneous ventricular assist device according to claim 15, wherein each mating connector further comprises a silicone gasket, the detent being positioned between the silicone gasket and the connector.

17. The percutaneous ventricular assist device according to claim 1, wherein each mating connector, each connector, or both comprises a magnetic element.

18. A system for ventricular assistance, comprising:
a percutaneous ventricular assist device according to claim 1; and
a controller configured to control the percutaneous ventricular assist device.

19. A kit comprising:
a pump assembly having one or more connectors at or near a distal portion;
a first cannula assembly having one or more mating connectors at or near a proximal portion of the first cannula assembly;
a second cannula assembly having one or more mating connectors at or near a proximal portion of the second cannula assembly, the second cannula assembly being different than the first cannula assembly;
wherein the one or more mating connectors of first cannula assembly engage with the one or more connector of the pump assembly such that the first cannula assembly is coupled to the pump assembly.

20-55. (canceled)

56. A percutaneous ventricular assist device, comprising:
a pump assembly having a distal end and proximal end;
a first cannula assembly having a first cannula and a sensor, the first cannula assembly having a distal end and a proximal end, the proximal end attached to the pump assembly, the distal end having one or more connectors;
a second cannula assembly having a second cannula and a first inflow cage, wherein the second cannula includes one or more mating connectors engageable with the one or more connectors of the first cannula assembly such that the second cannula assembly is attachable to the first cannula assembly by a clinician before a medical procedure.

57-72. (canceled)
